(19) United States
(12) Patent Application Publication
(10) Pub. No.: US 2011/0112470 A1
(43) Pub. Date: May 12, 2011

(54) SYRINGE

(75) Inventors: Christian Lingenfelder, Ulm (DE); Benjamin Knopp, Blaustein (DE)

(73) Assignee: FLUORON GMBH, Ulm (DE)

(21) Appl. No.: 12/943,625

(22) Filed: Nov. 10, 2010

(30) Foreign Application Priority Data
Nov. 10, 2009 (DE) 10 2009 052 552.1

(51) Int. Cl. A61M 13/00

(52) U.S. Cl. 604/26

(57) ABSTRACT

The invention relates to a syringe (1) having a syringe body (2) which has an interior space (3) and a tip (4) connected to the interior space (3). A plunger rod (5) is guided in the interior space (3) in a longitudinally displaceable manner. A syringe attachment filter (6) can be fixed on the tip (4), and a sealing stopper (7) can be secured on the syringe attachment filter (6).

### FIELD OF THE INVENTION

The invention relates to a syringe according to the preamble of claim 1.

In particular, the invention relates to a ready-to-use system of a gas tamponade for application in medicine. The tamponade is used to fill a natural cavity, for example a cavity that that has developed as a result of an ageing-related vitreous-body shrinkage, or an artificial cavity, for example a cavity that has developed as a result of a vitrectomy.

### BACKGROUND OF THE INVENTION

Ageing-related disorders, such as retinal changes or retinal detachments, glaucoma, cataract, and also ageing-related macular degeneration and diabetes-related retinopathy, are increasing owing to the rising life-expectancy of human beings. In order to treat these and also other disorders of the eye, mostly a vitrectomy (removal of the vitreous body) is required. The resultant hollow space must be refilled in order to prevent a collapse of the vitreous-body space. “Heavy gases”, such as SF₆, C₂F₆ or C₃F₈, are used for this purpose. Heavy gases are generally spoken of when the gases have a distinctly higher density when compared with normal ambient air.

The therapeutic effect in most cases does not develop as a result of the gas itself, but rather as a result of the gas-fluid interface. This surface tension precludes the passage of gas through a hole in the retina into the subretinal space, in addition the hole in the retina is relaxed, and further passage of fluid into the subretinal space is prevented.

After the injection of the heavy gas, a diffusion of O₂ and CO₂ starts from the blood into the eye, as a result of which the volume of the gas bubble increases. After a few hours, a diffusion equilibrium sets in for O₂ and CO₂, yet for N₂ only sets in after a few days.

The heavy gases are absorbed through the retina by way of the choroid membrane and depending on the type leave the eye within 1 to 2 weeks.

The gases used in surgery are in most cases transferred directly during the operation out of a steel cylinder into the medium that is to be used.

U.S. Pat. No. 6,866,142 B2 describes a system which consists of a disposable syringe that is already filled with gas and which for better gas-tightness is kept in a container that is filled with the same gas as the disposable syringe.

All of the systems set out above have a plurality of disadvantages.
In all the systems in part considerable quantities of SF₆, C₂F₆ and C₃F₈ are discharged into the environment, although these gases number among the strongest known greenhouse gases; thus, for example, 1 kg SF₆ has the same effects as 22.2 t. CO₂ [EC Regulation No. 842/2006].
Filling gas tamponades from the steel cylinder in the operating theatre is not permitted, since steel cylinders must be kept in a special steel cabinet for pressure vessels.
The single doses that are on the market must be prepared in a plurality of steps before they can be used by the surgeon.

It is therefore an object of the present invention to provide a syringe of the kind specified in the preamble of claim 1 that is able to remove the disadvantages of the prior art previously explained and in particular is simple to keep and operate.

This object is achieved by means of the features of claim 1.

The subclaims have advantageous further developments of the invention as their content.

### DETAILED DESCRIPTION OF THE INVENTION

The invention thus relates to a system with a disposable syringe, preferably 50 ml, with a Luer-lock extension, which is used as a gas container, with a syringe attachment filter that is screwed onto the syringe with a Luer-lock connection on both sides and with a Luer stopper in order to close the system.

The assembled system (disposable syringe and syringe attachment filter) is filled with a few millilitres of the respective gas and closed with the Luer stopper.

The system is preferably provided with a sticker which distinguishes in colour the mixture ratios for SF₆ (20% gas/80% air), C₂F₆ (16% gas/84% air) and C₃F₈ (12% gas/88% air), and also marks the maximum value up to which the plunger must/may be drawn out in order to adjust the mixture ratio. The closed system is packed, preferably in a sterile bag, and subsequently sterilized, preferably in a steam autoclave.

Further details, features and advantages of the invention emerge from the following description of exemplary embodiments with reference to the drawing.

### In the drawing

FIG. 1 shows a diagrammatically slightly simplified representation of a first embodiment of a syringe in accordance with the invention, and

FIG. 2 shows a representation, corresponding to FIG. 1, of a second embodiment of the syringe in accordance with the invention.

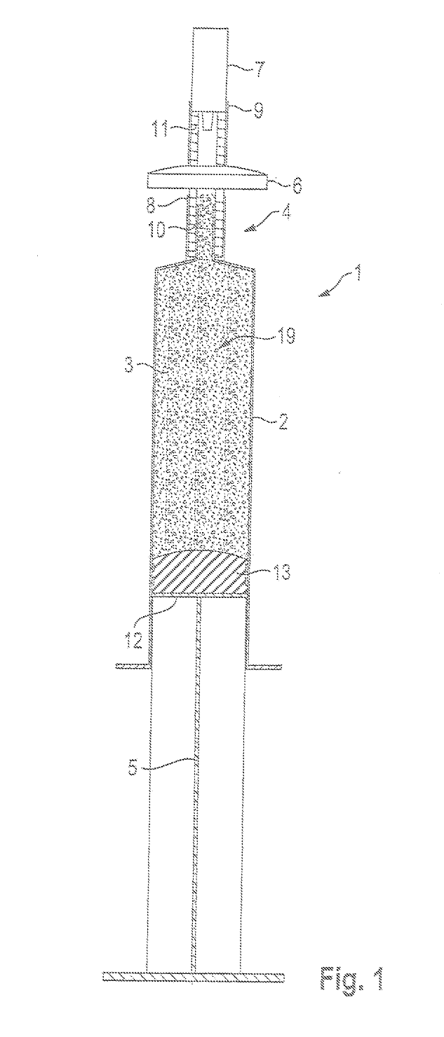

A first embodiment of a syringe 1 in accordance with the invention that has a cylindrical syringe body 2 enclosing an interior space 3 is shown in FIG. 1.

The interior space 3 is connected to a tip 4, and a plunger rod 5 is guided in the interior space 3 in a longitudinally displaceable manner.

As FIG. 1 illustrates, a syringe attachment filter 6 is fixed on the tip 4. The whole system is closed, furthermore, in a gas-tight manner by a sealing stopper 7 which in turn can be fixed on the outer end of the syringe attachment filter 6.

In the case of an embodiment that is particularly preferred, the syringe attachment filter 6 has a first fixing section 8 for mounting on the tip 4 and a second fixing section 9 for fastening the sealing stopper 7.

The syringe attachment filter 6 and the sealing stopper 7 can preferably be fixed by means of a respective screw connection 10 and 11. In the case of a particularly preferred embodiment, the screw connections 10 and 11 are each formed as Luer-lock closures.

As FIG. 1 illustrates furthermore, the plunger rod 5 has at its end region 12 that is arranged in the interior space 3 a rubber stopper 13, which is preferably silicone-treated.

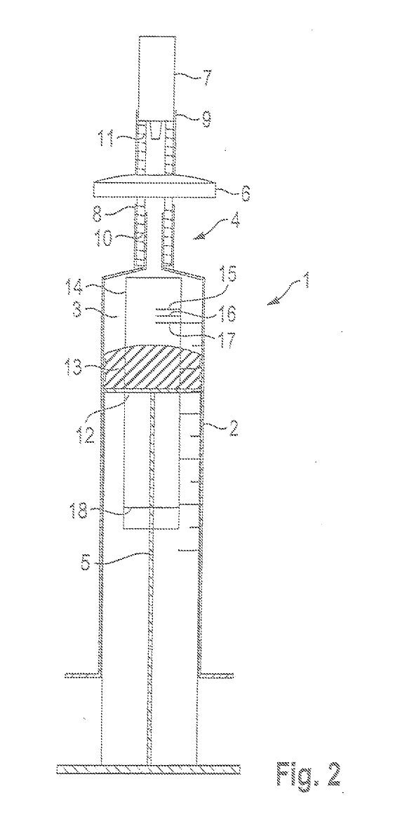

The embodiment in accordance with FIG. 2 corresponds, to the greatest possible extent, to that of FIG. 1 so all the corresponding features are provided with the same reference numerals as in FIG. 1. Reference may be made in this respect to the description of FIG. 1 given above.

The embodiment of the syringe 1 in accordance with FIG. 2 is distinguished by the fact that a label 14 is fixed on the syringe body 2. As FIG. 2 illustrates, the label 14 has marks 15, 16 and 17 for different mixture ratios between heavy gas 19 and the air that is to be taken in by suction. Furthermore, a mark 18 is provided for the maximum plunger stroke.

As already explained at the beginning, the syringe 1 can be formed as a disposable syringe.

The gas that is introduced in the interior space 3 of the syringe body 2 is preferentially a heavy gas, in particular SF₆, C₃F₈ or C₂F₆.

In addition to the written disclosure of the invention given above, reference is hereby made explicitly to the representation thereof in the drawings in FIGS. 1 and 2.

### LIST OF REFERENCE NUMERALS

1 Syringe
2 Syringe body
3 Interior space
4 Tip
5 Plunger rod
6 Syringe attachment filter
7 Sealing stopper
8 Fixing section
9 Fixing section
10, 11 Screw connections
13 Rubber stopper
14 Label
15, 16, 17 Mark
19 Gas

1. A syringe (1),
having a syringe body (2),
which has an interior space (3) and
which has a tip (4) connected to the interior space (3);
having a plunger rod (5) which is guided in the interior space (3) in a longitudinally displaceable manner, characterised in that
a syringe attachment filter (6) can be fixed on the tip (4), and
a sealing stopper (7) can be fixed on the syringe attachment filter (6).

2. A syringe according to claim 1, characterised in that the syringe attachment filter (6) has a first fixing section (8) for mounting on the tip (4) and a second fixing section (9) for fastening the sealing stopper (7).

3. A syringe according to claim 1, characterised in that the syringe attachment filter (6) and the sealing stopper (7) can be fixed by means of a respective screw connection (10 and 11).

4. A syringe according to claim 3, characterised in that the screw connection (10 and 11 respectively) is formed as a Luer-lock closure.

5. A syringe according to claim 1, characterised in that the plunger rod (5) is provided with a rubber stopper (13) at its end region (12) that is arranged in the interior space (3).

6. A syringe according to claim 5, characterised in that the rubber stopper (13) is silicone-treated.

7. A syringe according to claim 1, characterised in that a label (14) with mixture-ratio marks (15, 16 and 17) and a mark (18) for the maximum plunger stroke can be provided on the syringe body (2).

8. A syringe according to claim 1, characterised in that it is formed as a disposable syringe.

9. A syringe according to claim 1, characterised in that the syringe body (2) can be filled with a heavy gas (19).

10. A syringe according to claim 9, characterised in that the heavy gas is SF₆, C₃F₈ or C₂F₆.
